(19) United States
(12) Patent Application Publication
(10) Pub. No.: US 2022/0307921 A1
(43) Pub. Date: Sep. 29, 2022

(54) System and Method for Performing Contact Tracing Based on Temperature Measurements

(71) Applicant: AVX Antenna, Inc. d/b/a Ethertronics, Inc., San Diego, CA (US)

(72) Inventor: Laurent Desclos, San Diego, CA (US)

(21) Appl. No.: 17/704,212

(22) Filed: Mar. 25, 2022

Related U.S. Application Data
(60) Provisional application No. 63/167,438, filed on Mar. 29, 2021.

(51) Int. Cl. G01K 13/20; G01K 13/02; G08B 5/38; G08B 3/10

(52) U.S. Cl. CPC G01K 13/20; G01K 13/02; G08B 5/38; G08B 3/10

(57) ABSTRACT

A method of performing contact tracing of persons on a network is provided. The method includes obtaining temperature data comprising one or more temperature readings for a first person associated with a first sensor assembly of a plurality of sensor assemblies on the network. The method includes determining whether the one or more temperature readings for the first person exceeds a threshold temperature. Responsive to determining the one or more temperature readings exceeds the threshold temperature, the method includes determining, by the one or processors, whether a second sensor assembly of the plurality of sensor assemblies is within a predetermined proximity of the first sensor assembly. Responsive to determining the second sensor assembly is within the predetermined proximity of the first sensor assembly, the method includes providing a notification indicative of a second person associated with the second sensor assembly being within the predetermined proximity of the first person.

### PRIORITY CLAIM

The present application claims the benefit of priority of U.S. Provisional App. No. 63/167,438, titled “System and Method for Performing Contact Tracing Based on Temperature Measurements,” having a filing date of Mar. 29, 2021, which is incorporated by reference herein.

### FIELD

The present disclosure relates generally to a wireless network and, more particularly, to a system and method for performing contact tracing based on data obtained from sensor assemblies on the wireless network.

### BACKGROUND

A disease (e.g., virus) can be transmitted from one person to another through a variety of means (e.g., air, touch). Contact tracing can be used to identify one or more persons who have been exposed to an individual afflicted with the disease (e.g., virus). In this manner, the one or persons identified through contact tracing can be quarantined to prevent onward transmission of the disease. Data collected from a variety of sources can be used to perform contact tracing. For instance, data indicative of a location of person devices (e.g., smartphone) can be used to determine whether the one or more persons came in contact with the individual afflicted with the disease.

### SUMMARY

Aspects and advantages of embodiments of the present disclosure will be set forth in part in the following description, or may be learned from the description, or may be learned through practice of the embodiments.

In one aspect, a method of performing contact tracing of persons on a network is provided. The method includes obtaining temperature data comprising one or more temperature readings for a first person associated with a first sensor assembly of a plurality of sensor assemblies on the network. The method includes determining whether the one or more temperature readings for the first person exceeds a threshold temperature. Furthermore, in response to determining the one or more temperature readings exceeds the threshold temperature, the method includes determining, by the one or processors, whether a second sensor assembly of the plurality of sensor assemblies is within a predetermined proximity of the first sensor assembly. Still further, in response to determining the second sensor assembly is within the predetermined proximity of the first sensor assembly, the method includes providing a notification indicative of a second person associated with the second sensor assembly being within the predetermined proximity of the first person.

In another aspect, a system for performing contact tracing is provided. The system includes a plurality of sensor assemblies. Each of the sensor assemblies can be associated with a different person. Furthermore, one or more of the sensor assemblies can include a multi-mode antenna configurable in a plurality of antenna modes. Each of the antenna modes can include a distinct radiation pattern. The system further includes a network controller communicatively coupled to the plurality of sensor assemblies via a network. In response to determining one or more temperature readings for a first person associated with a first sensor assembly of the plurality of sensor assemblies exceeds a threshold temperature, the network controller is configured to determine whether a second sensor assembly of the plurality of sensor assemblies is within a predetermined proximity of the first sensor assembly. Furthermore, in response to determining the second sensor assembly is within the predetermined proximity of the first sensor assembly, the network controller is configured to provide a notification indicative of a second person associated with the second sensor assembly being within the predetermined proximity of the first person.

These and other features, aspects and advantages of various embodiments will become better understood with reference to the following description and appended claims. The accompanying drawings, which are incorporated in and constitute a part of this specification, illustrate embodiments of the present disclosure and, together with the description, serve to explain the related principles.

### BRIEF DESCRIPTION OF THE DRAWINGS

Detailed discussion of embodiments directed to one of ordinary skill in the art are set forth in the specification, which makes reference to the appended figures, in which:

FIG. 1 depicts a system for performing contact tracing according to example embodiments of the present disclosure.

FIG. 2 depicts a local area network of the system of FIG. 1 configured as a mesh network according to example embodiments of the present disclosure.

FIG. 3 depicts a multi-mode antenna according to example embodiments of the present disclosure.

FIG. 4 depicts a two-dimensional radiation pattern associated with a multi-mode antenna according to example embodiments of the present disclosure.

FIG. 5 depicts a frequency plot of a multi-mode antenna according to example embodiments of the present disclosure.

FIG. 6 depicts a block diagram of components of a sensor assembly according to example embodiments of the present disclosure.

FIG. 7 depicts a flow diagram of a method for performing contact tracing based on temperature data according to example embodiments of the present disclosure.

FIG. 8 depicts a flow diagram of a method for determining whether two sensor assemblies are within a predetermined proximity of one another according to example embodiments of the present disclosure.

FIG. 9 depicts another system for performing contact tracing according to example embodiments of the present disclosure.

### DETAILED DESCRIPTION

Reference now will be made in detail to embodiments, one or more examples of which are illustrated in the drawings. Each example is provided by way of explanation of the embodiments, not limitation of the present disclosure. In fact, it will be apparent to those skilled in the art that various modifications and variations can be made to the embodiments without departing from the scope or spirit of the present disclosure. For instance, features illustrated or described as part of one embodiment can be used with another embodiment to yield a still further embodiment. Thus, it is intended that aspects of the present disclosure cover such modifications and variations.

Example aspects of the present disclosure are directed to a system for performing contact tracing. The system can include a plurality of sensor assemblies on a wireless local area network. Each of the sensor assemblies can be associated with a different person. For instance, a first sensor assembly can be associated (e.g., worn by) with a first person. Conversely, a second sensor assembly can be associated with a second person. Each of the plurality of sensor assemblies can include one or more temperature sensors. In this manner, each of the plurality of sensor assemblies can obtain temperature data (e.g., one or more temperature readings) indicative of a temperature of a corresponding person.

In some implementations, one or more of the plurality of sensor assemblies can include a multi-mode antenna. The multi-mode antenna can be configurable in a plurality of antenna modes. Each of the plurality of antenna modes can have a distinct radiation pattern and/or polarization. For instance, in some implementations, the multi-mode antenna can be configurable in three different antenna modes. In alternative implementations, the multi-mode antenna can be configured in more or fewer antenna modes.

As discussed above, each of the sensor assemblies can include one or more temperature sensors. In this manner, the one or more temperature sensors of a first sensor assembly associated with a first person can obtain temperature data indicative of a temperature of the first person. For instance, the temperature data can include one or more temperature readings indicative of the temperature (e.g., body temperature) of the first person. Furthermore, the one or more temperature readings can be compared to a threshold temperature to determine whether the first person has an elevated temperature. For example, the threshold temperature can correspond to a temperature reading of about 100 degrees Fahrenheit). In this manner, the system can determine whether the first person is running a fever, which can be a symptom of a disease (e.g., viral).

When the first person has an elevated temperature, the system according to example embodiments of the present disclosure can determine whether other persons come within a predetermined proximity of the first person. For instance, the system can determine whether a second sensor assembly comes within the predetermined proximity of the first sensor assembly based, at least in part, on one or more channel quality indicators associated with a communication link between the first sensor assembly and the second sensor assembly. The one or more channel quality indicators can include, for instance, a receive signal strength indicator associated with a beacon signal that is transmitted via the multi-mode antenna of the second sensor assembly and received via the multi-mode antenna of the first sensor assembly.

In some implementations, one or more control devices of the first sensor assembly can configure the multi-mode antenna of the first sensor assembly in each of the plurality of antenna modes while the second sensor assembly is broadcasting the beacon signal. In this manner, the multi-mode antenna of the first sensor assembly can scan for the beacon signal while configured in one or more of the antenna modes. For instance, the multi-mode antenna of the first sensor assembly can receive the beacon signal while configured in a first antenna mode of the plurality of antenna modes. Conversely, the multi-mode antenna of the first sensor assembly may not receive the beacon signal when configured in a second antenna mode of the plurality of antenna modes.

In some implementations, the system can be configured to determine the second sensor assembly is within the predetermined proximity when the RSSI value of the beacon signal exceeds the threshold value (e.g., threshold signal strength). For instance, in some implementations, the threshold value can correspond to the RSSI value of the beacon signal when the second sensory assembly is within about 6 feet of the first sensor assembly. As used herein, use of the term “about” in conjunction with a stated numerical value refers to a range of numerical values within 10% of the stated numerical value.

Furthermore, when the system determines the second sensor assembly is within the predetermined proximity of the first sensor assembly, the system can generate one or more notifications indicative of a second person associated with the second sensor assembly being within the predetermined proximity (e.g., about 6 feet) of the first person. In some implementations, the one or more notifications can be provided to one or more output devices of the second sensor assembly that is associated with (e.g., worn) by the second person. For instance, the one or more output devices can include a light emitting diode (LED) configured to provide a visual alert (e.g., blinking light). Alternatively, or additionally, the one or more output devices can include a speaker configured to provide an audible alert. In this manner, the one or more notifications can alert the second person of the potential exposure to disease (e.g., virus) due, at least in part, to the second person coming within the predetermined proximity of the first person. Furthermore, the one or more notifications can prompt the second person to take one or more precautions (e.g., self-quarantine) to prevent further spreading of the disease.

The system according to example aspects of the present disclosure can provide numerous technical benefits and advantages. For instance, configuring the multi-mode antenna of the first sensor assembly in each of the plurality of antenna modes while the second sensor assembly is broadcasting the beacon signal can improve decision-making of the system regarding whether the second sensor assembly is within the predetermined proximity of the first sensor assembly. In this manner, contact tracing performed by the system can be improved since instances in which the second sensor assembly is erroneously determined to be outside the predetermined proximity of the first sensor assembly can be avoided.

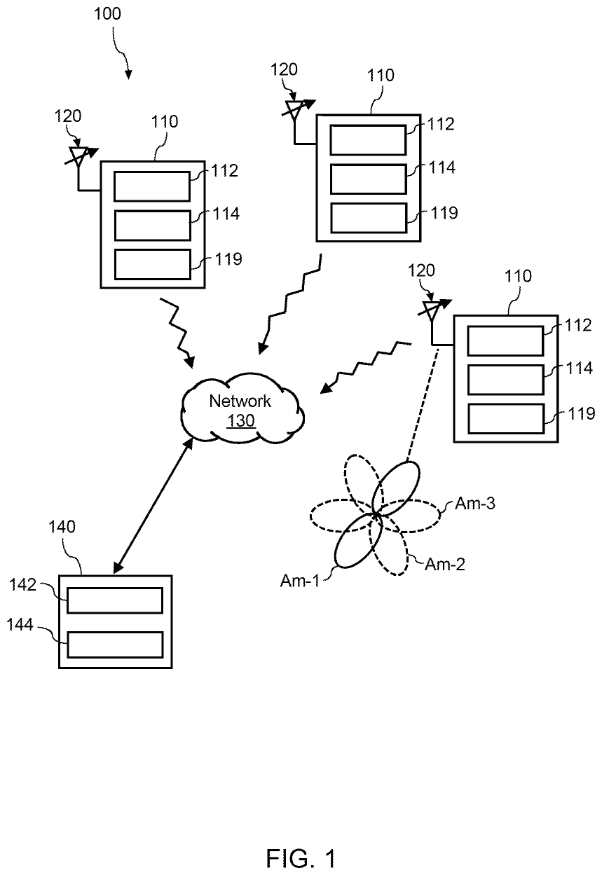

Referring now to the FIGS, FIG. 1 depicts a system 100 for performing contact tracing of persons according to example embodiments of the present disclosure. As shown, the system 100 can include a plurality of sensor assemblies 110. Each of the sensor assemblies 110 can be associated with a different person. For instance, a first sensor assembly can be associated with a first person, whereas a second sensor assembly can be associated with a second person. More particularly, the first sensor assembly and the second sensor assembly can be associated with the first person and the second person, respectively. Details of the sensor assemblies 110 will now be discussed in more detail.

Each of the sensor assemblies 110 can include one or more temperature sensors 112. For instance, in some implementations, each of the sensor assemblies 110 can include a first temperature sensor and a second temperature sensor. The first temperature sensor can be positioned at a first location (e.g., chest) on the person, whereas the second temperature sensor can be positioned at a second location (e.g., armpit) that is different than the first location. In this manner, the temperature sensors 112 (e.g., first temperature sensor and second temperature sensor) can obtain temperature measurements at multiple locations on the person's body.

In some implementations, one or more of the sensor assemblies can include a first temperature configured to measure a body temperature of the person. In addition, the one or more sensor assemblies can include a second temperature sensor configured to measure an ambient temperature of an environment surrounding the person. In this manner, the system 100 can be configured to adjust the body temperature reading of the person as needed based, at least in part, on the ambient temperature reading.

In some implementations, one or more of the sensor assemblies 110 can include a multi-mode antenna 120. The multi-mode antenna 120 can be configurable in a plurality of different antenna modes (e.g., Am−1, Am, 2, Am, 3, etc.). Each antenna mode of the plurality of antenna modes can be associated with a different radiation pattern and/or polarization. It should be understood that each of the sensor assemblies 110 can include any suitable number of multi-mode antennas 120. For instance, in some implementations, one or more of the sensor assemblies 110 can include two or more multi-mode antennas 120.

In some implementations, each of the sensor assemblies 110 can include one or more control devices 114. The one or more control devices 114 can be configured to obtain temperature data from the one or more temperature sensors 112. It should be understood that temperature data can include one or more temperature readings for a person associated with the corresponding sensor assembly 110. In some implementations, the one or more control devices 114 can be configured to compare the temperature data to one or more threshold temperatures. For example, in some implementations, the one or more threshold temperatures can include a temperature reading indicative of a person having a fever.

In some implementations, the one or more control devices 114 can be configured to control operation of the multi-mode antenna 120. For instance, the one or more control devices 114 of a first sensor assembly can configure the multi-mode antenna 120 of the first sensor assembly in one of the plurality of antenna modes (e.g., Am−1, Am−2, Am−3, etc.) to facilitate communication with a second sensor assembly.

In some implementations, one or more of the sensor assemblies 110 can include one or more output devices 119. For instance, the one or more output devices 119 can include one or more light emitting diodes (LEDs). Alternatively, or additionally, the one or more output devices 119 can include one or speakers. In this manner, the one or more output devices 119 can output notifications (e.g., audible and/or visual) to notify the person associated with the corresponding sensor assembly 110 of a condition (e.g., elevated temperature, exposure to another person having an elevated temperature, etc.).

In some implementations, the sensor assemblies 110 can communicate with one another via a wireless local area network 130. The wireless local area network 130 can be any suitable type of network or combination of networks that allows for communication between the sensor assemblies 110. In some implementations, the wireless local area network 130 can include one or more of a secure network, Wi-Fi network, IoT network, mesh network, one or more peer-to-peer communication links, and/or some combination thereof, and can include any number of wired or wireless links. Communication over the wireless local area network 130 can be accomplished, for instance, via a communication interface using any type of protocol, protection scheme, encoding, format, packaging, etc.

In some implementations, the system 100 can include a network controller 140. The network controller 140 can be communicatively coupled to the plurality of sensor assemblies 110 via the wireless local area network 130. As shown, the network controller 140 can include one or more processors 142 and one or more memory devices 144. The processor(s) 142 can include any suitable processing device, such as a microprocessor, microcontroller, integrated circuit, logic device, or other suitable processing device. The memory device(s) 144 can include one or more computer-readable media, including, but not limited to, non-transitory computer-readable media, RAM, ROM, hard drives, flash drives, or other memory devices. The memory device(s) 144 can store information accessible by the processor(s) 142, including computer-readable instructions that can be executed by the processor(s) 142. The computer-readable instructions can be any set of instructions that, when executed by the processor(s) 142, cause the processor(s) 142 to perform operations. The computer-readable instructions can be software written in any suitable programming language or may be implemented in hardware.

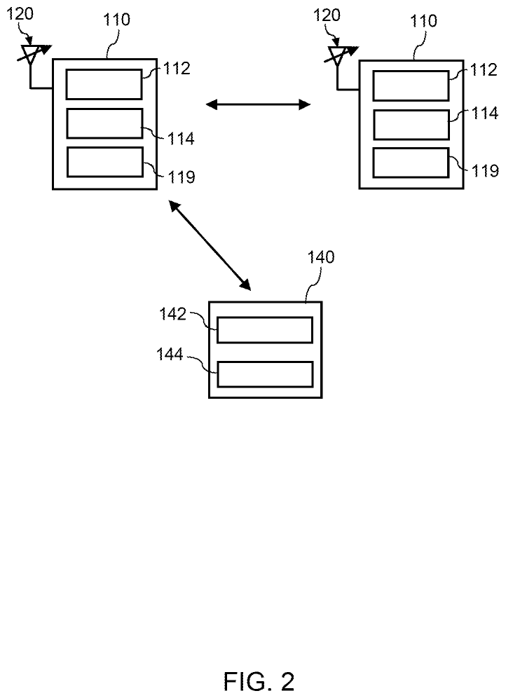

Referring now to FIG. 2, the wireless local area network 130 can, in some implementations, be configured as a wireless mesh network. In the wireless mesh network, a first sensor assembly 150 of the plurality of sensor assemblies 110 (FIG. 1) can be in direct communicate with the network controller 140. In this manner, the first sensor assembly 150 can transmit data directly to the network controller 140. Conversely, a second sensor assembly 152 of the plurality of sensor assemblies 110 (FIG. 1) can be in communication with the network controller 140 via the first sensor assembly 150. In this manner, the second sensor assembly 152 can transmit data to the network controller 140 via the first sensor assembly 150.

In such implementations, the network controller 140 can be configured to dynamically reconfigure the wireless mesh network based, at least in part, on data indicative of one or more channel quality indicator associated with each of the plurality of communication links in the wireless mesh network. Examples of the one or more channel quality indicators can include, for instance, a signal to interference noise ratio (SINR), a receive signal strength indicator (RSSI), a modulation coding scheme (MCS). In this manner, the network controller 140 can reconfigure communication links on the wireless mesh network as needed to load balance traffic on the wireless mesh network.

As an example, the network controller 140 can reconfigure the wireless mesh network such that the second sensor assembly 152 communicates directly with the network controller 140 instead of indirectly via the first sensor assembly 150. Alternatively, or additionally, the network controller 140 can reconfigure the wireless mesh network such that the first sensor assembly 150 no longer communicates directly with the network controller 140. Instead, the network controller 140 can reconfigure the wireless mesh network such that the first sensor assembly 150 is in communication with the network controller 140 via another sensor assembly 110 (FIG. 1) of the plurality of sensor assemblies 110.

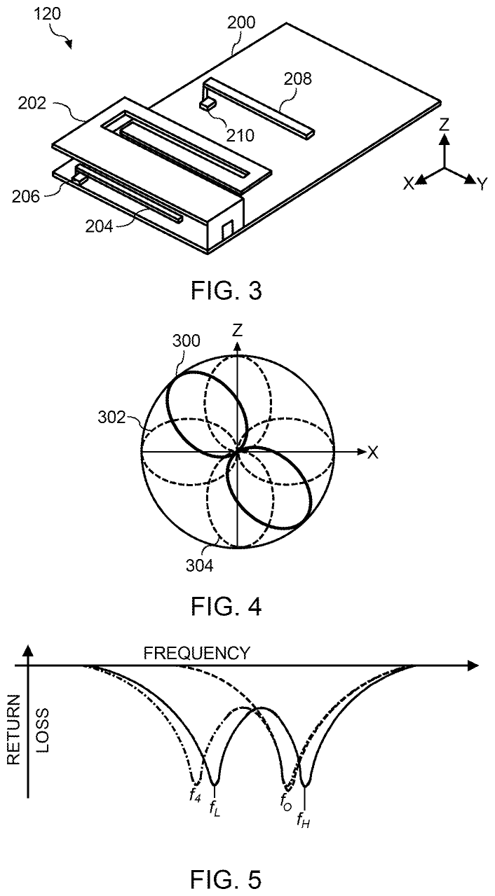

FIG. 3 illustrates an example multi-mode antenna 120 according to the present disclosure. As shown, the multi-mode antenna 120 can include a circuit board 200 (e.g., including a ground plane) and a driven antenna element 202 disposed on the circuit board 200. An antenna volume may be defined between the circuit board 200 (e.g., and the ground plane) and the driven antenna element 202. The multi-mode antenna 120 can include a first parasitic element 204 positioned at least partially within the antenna volume. The multi-mode antenna 120 can further include a first tuning element 206 coupled with the first parasitic element 204. The first tuning element 206 can be a passive or active component or series of components and can be configured to alter a reactance on the first parasitic element 204 either by way of a variable reactance or shorting to ground. It should be appreciated that altering the reactance of the first parasitic element 204 can result in a frequency shift of the multi-mode antenna 120. It should also be appreciated that the first tuning element 206 can include at least one of a tunable capacitor, MEMS device, tunable inductor, switch, a tunable phase shifter, a field-effect transistor, or a diode.

In some implementations, the multi-mode antenna 120 can include a second parasitic element 208 disposed adjacent the driven antenna element 202 and outside of the antenna volume. The multi-mode antenna 120 can further include a second tuning element 210. In some implementations, the second tuning element 210 can be a passive or active component or series of components and may be configured to alter a reactance on the second parasitic element 208 by way of a variable reactance or shorting to ground. It should be appreciated that altering the reactance of the second parasitic element 208 result in a frequency shift of the multi-mode antenna 120. It should also be appreciated that the second tuning element 210 can include at least one of a tunable capacitor, MEMS device, tunable inductor, switch, a tunable phase shifter, a field-effect transistor, or a diode.

In example embodiments, operation of at least one of the first tuning element 206 and the second tuning element 210 can be controlled to adjust (e.g., shift) the antenna radiation pattern of the driven antenna element 202. For example, a reactance of at least one of the first tuning element 206 and the second tuning element 210 can be controlled to adjust the antenna radiation pattern of the driven antenna element 202. Adjusting the antenna radiation pattern can be referred to as “beam steering”. However, in instances where the antenna radiation pattern includes a null, a similar operation, commonly referred to as “null steering”, can be performed to shift the null to an alternative position about the driven antenna element 202 (e.g., to reduce interference).

FIG. 4 depicts antenna radiation patterns associated with the multi-mode antenna 120 of FIG. 1 according to example embodiments of the present disclosure. It should be appreciated that operation of at least one of the first parasitic element 204 and the second parasitic element 208 can be controlled to configure the multi-mode antenna 120 in a plurality of modes. It should also be appreciated that the multi-mode antenna 120 can have a distinct antenna radiation pattern or antenna polarization when configured in each of the plurality of modes.

In some implementations, the multi-mode antenna 120 can have a first antenna radiation pattern 300 when the multi-mode antenna 120 is configured in a first mode of the plurality of modes. In addition, the multi-mode antenna 120 can have a second antenna radiation pattern 302 when the multi-mode antenna 120 is configured in a second mode of the plurality of modes. Furthermore, the multi-mode antenna 120 can have a third antenna radiation pattern 304 when the multi-mode antenna 120 is configured in a third mode of the plurality of modes. As shown, the first antenna radiation pattern 300, the second antenna radiation pattern 302, and the third antenna radiation pattern 304 can be distinct from one another. In this manner, the multi-mode antenna 120 can have a distinct radiation pattern when configured in each of the first mode, second mode, and third mode.

FIG. 5 depicts an example frequency plot of the multi-mode antenna 120 of FIG. 1 according to some aspects of the present disclosure. It should be understood that an electrical characteristic (e.g., reactance) of at least one of the first parasitic element 204 and the second parasitic element 208 can be controlled. In this manner, the electrical characteristic of at least one of the first parasitic element 204 and the second parasitic element 208 can be adjusted to shift a frequency at which the corresponding multi-mode antenna is operating.

In some implementations, the multi-mode antenna 120 can be tuned to a first frequency f₀ when the first parasitic element 204 and the second parasitic element 208 are deactivated (e.g., switched off). Alternatively and/or additionally, the multi-mode antenna 120 can be tuned to frequencies f_{L} and f_{H} when the second parasitic element 208 is shorted to ground. Furthermore, the multi-mode antenna 120 can be tuned to frequency f₄ when both the first parasitic element 204 and the second parasitic element 208 are shorted to ground. Still further, the multi-mode antenna 120 can be tuned to frequencies f₄ and f₀ when the first parasitic element 204 and the second parasitic element 208 are each shorted to ground. It should be understood that other configurations are within the scope of this disclosure. For example, more or fewer parasitic elements may be employed. The positioning of the parasitic elements may be altered to achieve additional modes that may exhibit different frequencies and/or combinations of frequencies.

FIGS. 3-5 depict one example of the multi-mode antenna 120 having a plurality of modes for purposes of illustration and discussion. Those of ordinary skill in the art, using the disclosures provided herein, will understand that other multi-mode antennas and/or antenna configurations can be used without deviating from the scope of the present disclosure. As used herein a “multi-mode antenna” refers to an antenna capable of operating in a plurality of modes where each mode is associated with a distinct radiation pattern.

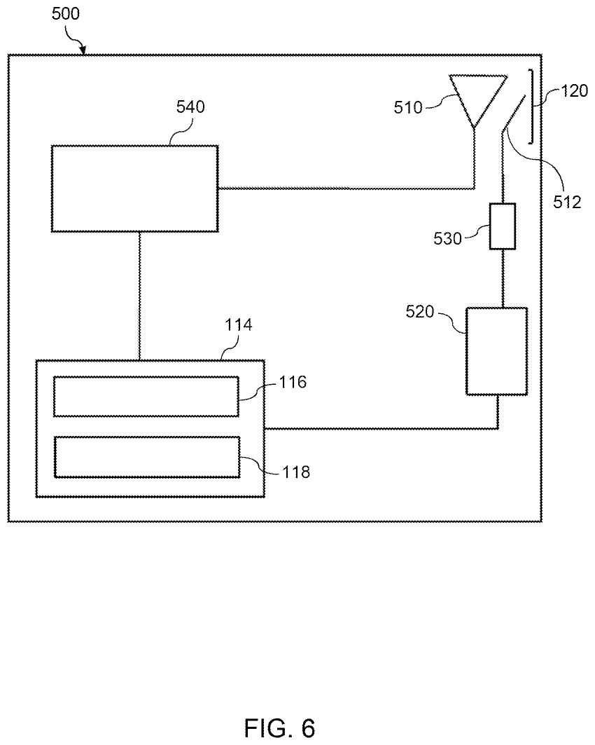

Referring now to FIG. 6, an example embodiment of a sensor assembly 500 is provided. As shown, the multi-mode antenna 120 can include a driven element 510 and a parasitic element 512. The multi-mode antenna 120 can, as discussed above, be operable in a plurality of different modes. Each mode of the plurality of modes can be associated with a different radiation pattern and/or polarization characteristics, for instance, as described above with reference to FIGS. 3-5. Furthermore, although the sensor assembly 500 is depicted as having only one multi-mode antenna 120, it should be appreciated that the sensor assembly 500 can include any suitable number of multi-mode antennas. For instance, in some implementations, the sensor assembly 500 can include two or more multi-mode antennas.

The sensor assembly 500 can include a tuning circuit 520 configured to control an electrical characteristic associated with the parasitic element 512 to operate the multi-mode antenna 120 in the plurality of different modes. In some implementations, the sensor assembly 500 can include a tunable component 530. As shown, the tunable component 530 can be coupled between the parasitic element 512 and the tuning circuit 520. The tuning circuit 520 can be configured to control operation of the tunable component 530 to alter the electrical connectivity of the parasitic element 512 with a voltage or current source or sink, such as coupling the parasitic element 512 to an electrical ground.

The sensor assembly 500 can include RF circuitry 540. In some implementations, the RF circuitry 540 can include a front end module. The front end module can include, for instance, one or more power amplifiers, low noise amplifiers, impedance matching circuits, etc. In this manner, the front end module can be configured to amplify the RF signal that is transmitted to and/or received from the driven element 510 of the multi-mode antenna 120.

In some implementations, the one or more control devices 114 of the sensor assembly 500 can be operatively coupled to the tuning circuit 520. In this manner, the one or more control devices 114 can be configured to control operation of the tuning circuit 520 to configure the multi-mode antenna 120 in the plurality of different modes. Alternatively, or additionally, the one or more control devices 114 can be in electrical communication with the RF circuitry 540. In this manner, RF signals received at the multi-mode antenna 120 can be provided to the one or more control devices 114 via the RF circuitry 540. In addition, the one or more control devices 114 can provide data to be modulated onto a transmit RF signal provided to the driven element 510 of the multi-mode antenna 120 via the RF circuitry 540.

As shown, the one or more control devices 114 can include one or more processors 116 and one or more memory devices 118. The processor(s) 116 can include any suitable processing device, such as a microprocessor, microcontroller, integrated circuit, logic device, or other suitable processing device. The memory device(s) 118 can include one or more computer-readable media, including, but not limited to, non-transitory computer-readable media, RAM, ROM, hard drives, flash drives, or other memory devices.

The memory device(s) 118 can store information accessible by the processor(s) 1116, including computer-readable instructions that can be executed by the processor(s) 132. The computer-readable instructions can be any set of instructions that, when executed by the processor(s) 116, cause the processor(s) 116 to perform operations. The computer-readable instructions can be software written in any suitable programming language or may be implemented in hardware.

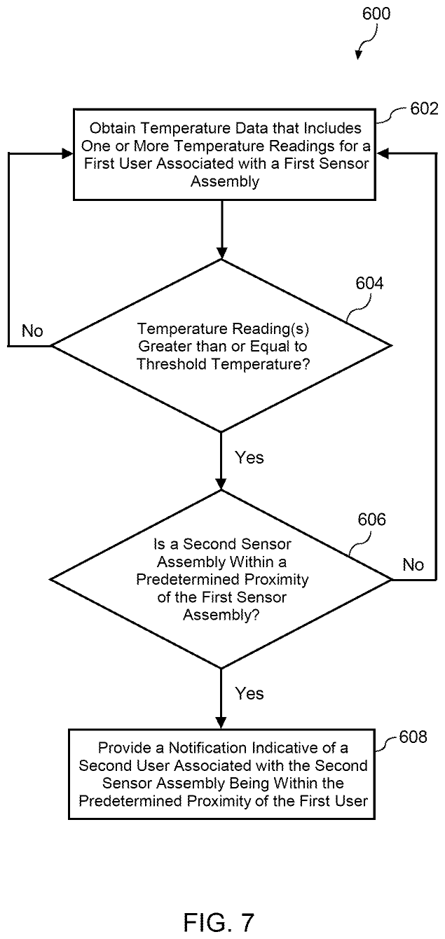

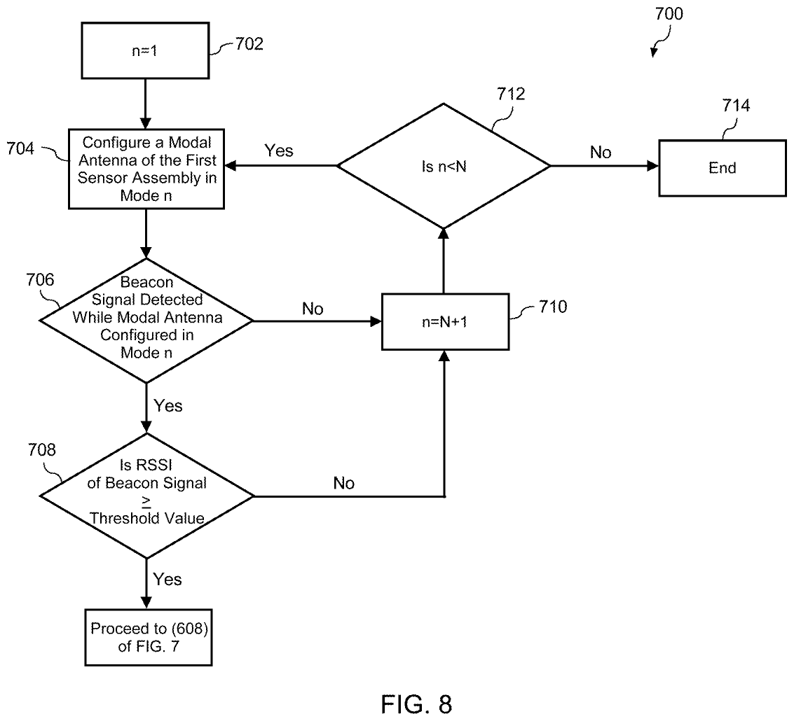

Referring now to FIG. 7, a flow diagram of a method 600 for configuring a multi-mode antenna onboard one or more of a plurality of network devices on a wireless network is provided according to example embodiments of the present disclosure. In general, the method 600 will be discussed herein with reference to the system 100 described above with reference to FIG. 1. For instance, in some implementations, the method 600, or a portion thereof , can be implemented by the one or more control devices 114 (FIG. 1) of one of the sensor assemblies (also FIG. 1). In alternative implementations, the method 600, or a portion thereof, can be implemented by the network controller 140 (FIG. 1). Additionally, although FIG. 7 depicts steps performed in a particular order for purposes of illustration and discussion, the method discussed herein is not limited to any particular order or arrangement. One skilled in the art, using the disclosure provided herein, will appreciate that various steps of the method disclosed herein can be omitted, rearranged, combined, and/or adapted in various ways without deviating from the scope of the present disclosure.

At (602), the method 600 includes obtaining temperature data for a first person associated with a first sensor assembly of a plurality of sensor assemblies on the network. The temperature data can include one or more temperature readings for the first person. For instance, in some implementations, the temperature data can include a first temperature reading and a second temperature reading. The first temperature reading can be obtained via a first temperature sensor of the first sensor assembly and can be indicative of a body temperature of the first person. The second temperature reading can be obtained via a second temperature sensor of the first sensor assembly and can be indicative of an ambient temperature of an environment surrounding the first person.

At (604), the method 600 includes determining whether the one or more temperature readings for the first person exceed a threshold temperature. For instance, in some implementations, the threshold temperature can correspond to a temperature reading indicative of a person having a fever. When the one or more temperature readings equal or exceed the threshold temperature, the method 600 proceeds to (606). Otherwise, the method 600 reverts to (602).

At (606), the method 600 includes determining whether a second sensor assembly of the plurality of sensor assemblies on the network is within a predetermined proximity of the first sensor assembly. For instance, the predetermined proximity can correspond to a distance needed between the second sensor assembly and the first sensor assembly to avoid transmission of the disease from the first person to the second person. In some implementations, the distance can be about 6 feet. When the second sensor assembly is within the predetermined proximity of the first sensor assembly, the method 600 proceeds to (608). Otherwise, the method 600 reverts (602) to determine whether the temperature of the first person is still elevated (e.g., above the threshold temperature).

At (608), the method 600 includes providing a notification indicative of a second person associated with the second sensor assembly being within the predetermined proximity of the first sensor assembly. The notification can include at least one of an audible notification or a visual notification. For instance, in some implementations,

Referring now to FIG. 8, a flow diagram of a method 700 for determining whether a second sensor assembly is within a predetermined proximity of the first sensor assembly is provided according to example embodiments of the present disclosure. In general, the method 700 will be discussed herein with reference to the system 100 described above with reference to FIG. 1. In addition, although FIG. 8 depicts steps performed in a particular order for purposes of illustration and discussion, the method discussed herein is not limited to any particular order or arrangement. One skilled in the art, using the disclosure provided herein, will appreciate that various steps of the method disclosed herein can be omitted, rearranged, combined, and/or adapted in various ways without deviating from the scope of the present disclosure.

At (702), the method 700 can include initializing a mode counter variable, n. For instance, the mode counter variable, n, can be initialized such that the mode counter variable, n, has an initial value of 1. At (704), the method 700 can include configuring the multi-mode antenna of the first sensor assembly in antenna mode n of a plurality of antenna modes N in which the multi-mode antenna is configurable.

At (706), the method 700 can include determining whether a beacon signal is received while the multi-mode antenna of the first sensor assembly is configured in mode n. When the first sensor assembly receives the beacon signal, the method 700 proceeds to (708). Otherwise, the method 700 proceeds to (710).

In some implementations, the beacon signal can include data identifying the second sensor assembly. For instance, in some implementations, the data identifying the second sensor assembly can include a medium access control (MAC) address that is unique to the second sensor assembly. In this manner, the MAC address can be used to determine which sensor assembly is transmitting the beacon signal.

At (708), the method 700 can include determining whether a received signal strength indicator (RSSI) value associated with the beacon signal equals or exceeds a threshold value. It should be understood that the threshold value can correspond to an RSSI value indicative of the second sensor assembly being within the predetermined proximity (e.g., about 6 feet) of the first sensor assembly. When the RSSI value associated with the beacon signal equals or exceeds the threshold value, the method 700 proceeds to (608) of the method 600 depicted in FIG. 6. Otherwise, the method 700 proceeds to (710).

At (710), the method 700 can include incrementing the mode counter variable, n. Furthermore, once the mode counter variable, n, has been incremented at (710), the method 700 can proceed to (712). At (712), the method 700 can include determining whether the current value of the mode counter variable, n, is greater than N, which corresponds to the total number of antenna modes in which the multi-mode antenna of the first sensor assembly is configurable. When the current value of the mode counter variable, n, is less than N, the method 700 reverts to (702). Otherwise, the method 700 proceeds to (714). At (714), the method can end. Alternatively, at (714), the method 700 can revert to (702).

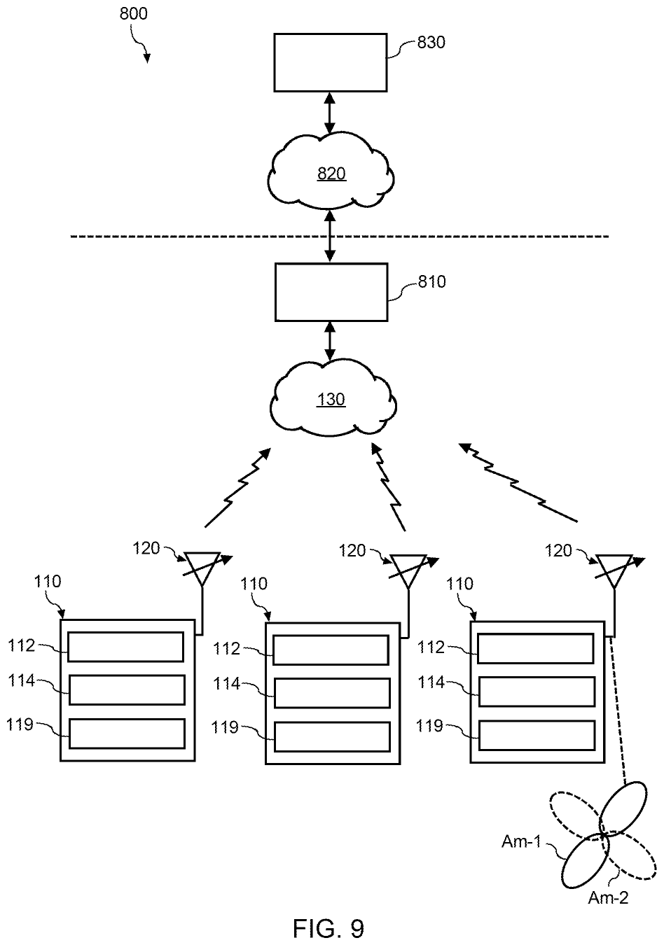

Referring now to FIG. 9, another system 800 for performing contact tracing of persons is provided according to example embodiments of the present disclosure. The system 800 can be configured in a similar manner to the system 100 discussed above with reference to FIG. 1. For instance, the system 800 depicted in FIG. 9 can include the plurality of sensor assemblies 110. However, in contrast to the system 100 of FIG. 1, the system 800 of FIG. 9 includes a gateway 810 that can allow access to a wide area network 820. The wide area network 820 can be, for instance, the Internet, cellular network, or other network, and can include any number of wired or wireless links. Communication over the wide area network 820 can be accomplished, for instance, via a communication interface using any type of protocol, protection scheme, encoding, format, packaging, etc. As shown, the sensor assemblies 110 can communicate information over the wide area network 820 to a remote computing system 830 via the gateway 810.

In some implementations, the gateway 810 can include a person device (e.g., smartphone, tablet, etc.) associated with each person of the system. For instance, a person device of a first person associated with a first sensor assembly of the system 800 can facilitate communication between the first sensor assembly and the remote computing system 830. Conversely, a person device of a second person associated with a second sensor assembly of the system 800 can facilitate communication between the second sensor assembly and the remote computing system 830.

The remote computing system 830 can include one or more computing devices. The one or more computing devices can include one or more processors and one or more memory devices. The remote computing system 830 can be distributed such that its components are located in different geographic areas. The technology discussed herein refers to computer-based systems and actions taken by and information sent to and from computer-based systems. One of ordinary skill in the art will recognize that the inherent flexibility of computer-based systems allows for a great variety of possible configurations, combinations, and divisions of tasks and functionality between and among components. For instance, processes discussed above may be implemented using a single computing device or multiple computing devices working in combination. Databases, memory, instructions, and applications may be implemented on a single system or distributed across multiple systems. Distributed components may operate sequentially or in parallel.

While the present subject matter has been described in detail with respect to specific example embodiments thereof, it will be appreciated that those skilled in the art, upon attaining an understanding of the foregoing may readily produce alterations to, variations of, and equivalents to such embodiments. Accordingly, the scope of the present disclosure is by way of example rather than by way of limitation, and the subject disclosure does not preclude inclusion of such modifications, variations and/or additions to the present subject matter as would be readily apparent to one of ordinary skill in the art.

What is claimed is:

1. A method of performing contact tracing of persons on a network, the method comprising:
obtaining, by one or more processors, temperature data comprising one or more temperature readings for a first person associated with a first sensor assembly of a plurality of sensor assemblies on the network, one or more of the sensor assemblies comprising a multi-mode antenna configurable in a plurality of antenna modes, each of the plurality of antenna modes having a distinct radiation pattern;
determining, by the one or more processors, whether the one or more temperature readings for the first person exceeds a threshold temperature;
responsive to determining the one or more temperature readings exceeds the threshold temperature, determining, by the one or processors, whether a second sensor assembly of the plurality of sensor assemblies is within a predetermined proximity of the first sensor assembly; and
responsive to determining the second sensor assembly is within the predetermined proximity of the first sensor assembly, providing, by the one or more processors, a notification indicative of a second person associated with the second sensor assembly being within the predetermined proximity of the first person.

2. The method of claim 1, wherein determining whether the second sensor assembly is within the predetermined proximity of the first sensor assembly comprises:
configuring, by the one or more processors, the multi-mode antenna of the first sensor assembly in each of the plurality of antenna modes;
obtaining, by the one or more processors, a beacon signal from at least the second sensor assembly while the multi-mode antenna of the first sensor assembly is configured in each of the plurality of antenna modes; and
determining, by the one or more processors, whether the second sensor assembly is within the predetermined proximity of the first sensor assembly based, at least in part, on the beacon signal.

3. The method of claim 2, wherein determining whether the second sensor assembly is within the predetermined proximity of the first sensor assembly comprises determining, by the one or more processors, whether a receive signal strength indicator associated with the beacon signal exceeds a threshold signal strength.

4. The method of claim 3, wherein determining whether the second sensor assembly is within the predetermined proximity of the first sensor assembly further comprises:
responsive to determining the receive signal strength indicator associated with the beacon signal obtained from the second sensor assembly while the first sensor assembly is configured in each of the plurality of antenna modes is below the threshold signal strength, determining, by the one or more processors, the second sensor assembly is not within the predetermined proximity of the first sensor assembly.

5. The method of claim 3, wherein determining whether the second sensor assembly is within the predetermined proximity of the first sensor assembly further comprises:
responsive to determining the receive signal strength indicator associated with the beacon signal obtained from the second sensor assembly while the multi-mode antenna of the first sensor assembly is configured in one of the plurality of antenna modes is above the threshold signal strength, determining, by the one or more processors, the second sensor assembly is within the predetermined proximity of the first sensor assembly.

6. The method of claim 1, wherein the threshold temperature is about 100 degrees Fahrenheit.

7. The method of claim 2, wherein the beacon signal comprises data indicative of an identifier that is unique to the second sensor assembly.

8. The method of claim 7, wherein the data indicative of the identifier comprises a medium access control (MAC) address.

9. The method of claim 1, wherein the notification comprises at least one of an audible notification or a visual notification.

10. The method of claim 1, wherein providing the notification indicative of the second person associated with the second sensor assembly being within the predetermined proximity of the first person comprises providing, by the one or more processors, the notification to the second sensor assembly.

11. A system for performing contact tracing, the system comprising:
a plurality of sensor assemblies, each of the plurality of sensor assemblies associated with a different person, each of the plurality of sensor assemblies comprising one or more temperature sensors, one or more of the plurality of sensor assemblies further comprising a multi-mode antenna configurable in a plurality of antenna modes, each of the plurality of antenna modes having a distinct radiation pattern; and
a network controller communicatively coupled to the plurality of sensor assemblies via a network, the network controller configured to perform operations, the operations comprising:
responsive to determining one or more temperature readings for a first person associated with a first sensor assembly of the plurality of sensor assemblies exceeds a threshold temperature, determining whether a second sensor assembly of the plurality of sensor assemblies is within a predetermined proximity of the first sensor assembly; and
responsive to determining the second sensor assembly is within the predetermined proximity of the first sensor assembly, providing a notification indicative of a second person associated with the second sensor assembly being within the predetermined proximity of the first person.

12. The system of claim 11, wherein the one or more temperature sensors comprise:
a first temperature sensor configured to obtain a first temperature reading indicative of a body temperature of a person; and
a second temperature sensor configured to obtain a second temperature reading indicative of an ambient temperature of an environment surrounding the person.

13. The system of claim 11, wherein determining whether the second sensor assembly is within the predetermined proximity of the first sensor assembly comprises:
configuring the multi-mode antenna of the first sensor assembly in each of the plurality of antenna modes;
obtaining a beacon signal from the second sensor assembly while the multi-mode antenna of the first sensor assembly is configured in each of the plurality of antenna modes; and
determining whether the second sensor assembly is within the predetermined proximity of the first sensor assembly based, at least in part, on the beacon signal.

14. The system of claim 13, wherein determining whether the second sensor assembly is within the predetermined proximity of the first sensor assembly comprises determining whether a receive signal strength indicator associated with the beacon signal exceeds a threshold signal strength.

15. The system of claim 11, wherein the plurality of sensor assemblies are communicatively coupled to one another via the network.

16. The system of claim 15, wherein the network comprises a local area network.

17. The system of claim 11, wherein providing the notification indicative of the second person associated with the second sensor assembly being within the predetermined proximity of the first person comprises providing the notification to one or more output devices of the second sensor assembly.

18. The system of claim 17, wherein the notification comprises at least one of an audible notification or a visual notification.
